(19) United States
(12) Patent Application Publication
(10) Pub. No.: US 2002/0086442 A1
(43) Pub. Date: Jul. 4, 2002

(54) Amino acid sequence determination for protein or the like

(75) Inventor: Hideshi Fujiwake, Kyoto (JP)

Correspondence Address: ARMSTRONG,WESTERMAN & HATTORI, LLP, 1725 K STREET, NW., SUITE 1000, WASHINGTON, DC, 20006, US

(73) Assignee: Shimadzu Corporation, Kyoto (JP)

(21) Appl. No.: 10/015,662

(22) Filed: Dec. 17, 2001

(30) Foreign Application Priority Data
Dec. 28, 2000 (JP) 2000-402767

(51) Int. Cl. G01N 33/543

(52) U.S. Cl. 436/518; 436/89

(57) ABSTRACT

Respective monoclonal antibodies are fixed into respective wells of a microplate one by one, and bonded with labeled analogs of PTH amino acid derivatives. A solution containing PTH amino acid derivatives obtained by Edman degradation is dripped on to the microplate for causing competitive reaction to the monoclonal antibodies with the analogs, and thereafter non-bonded PTH amino acid derivatives and liberated analogs are washed out. Then enzyme-labeled antibodies against the analogs are added and the quantity of bonded enzyme-labeled antibodies is measured thereby determining the types and quantities of the PTH amino acid derivatives. Constitutive amino acid can be detected in high sensitivity.

### BACKGROUND OF THE INVENTION

1. Field of the Invention

The present invention relates to a field for determining the amino acid sequence of protein or peptide.

2. Description of the Prior Art

A method of chemically cutting (Edman degradation) protein and/or peptide one by one from an N end side and determining constitutive amino acid has been established in the 1950s, and presently is still widely utilized. A current technique put into practice is a method of identifying constitutive amino acid liberated after Edman degradation as PTH amino acid (3-phenyl-2-thiohydantoin derivative) by high-speed liquid chromatography.

The current aforementioned method put into practice has the following problems:

1) Detection Sensitivity:

In UV (ultraviolet) detection type high-speed liquid chromatography, the limit of detection sensitivity for PTH amino acid is about 1 pmol to 100 fmol (as PTH amino acid). While a method utilizing fluorescence detection for fluorescent amino acid derivatives, a method utilizing mass spectrometry as detection means and the like have been proposed in order to solve this problem, none of these methods has been put into practice.

2) Identification in a case where constitutive amino acid is modified with saccharides or the like:

In a method utilizing the existing UV detection type high-speed liquid chromatography, liberated constitutive amino acid is identified from a retention time of a high-speed liquid chromatograph. However, in a case of the constitutive amino acid being modified with saccharides or by phosphorylation, the retention time is different from that for non-modified constitutive amino acid, and it is impossible to identify the constitutive amino acid or determine the type of the modification from the retention time.

Even if various types of modified PTH amino acid (e.g., phosphorylated PTH tyrosine) can be prepared as standard samples, it is necessary to set analytic conditions of the high-speed liquid chromatography so that respective retention times are different from each other in the method utilizing the current high-speed liquid chromatography. However, this is impossible in practice due to the large number of various types of modified PTH amino acid.

When utilizing high-speed liquid chromatography/mass spectrometry which is an analysis method of analyzing respective components separated by high-speed liquid chromatography further by mass spectrometry for also acquiring information on molecular weights, this is advantageous as to the problem of modification over analysis performed through only high-speed liquid chromatography due to the addition of the information on the molecular weights. However, sensitivity is still insufficient as described in the above item 1).

### SUMMARY OF THE INVENTION

Accordingly, a first object of the present invention is to make it possible to detect liberated constitutive amino acid in higher sensitivity than a conventional method.

A second object of the present invention is to make it possible to detect modified liberated constitutive amino acid in high sensitivity.

The present invention is directed to an amino acid sequence determination for protein or peptide including the following steps (A) and (B):

(A) chemically cutting constitutive amino acid one by one from an N end of protein or peptide for liberating the constitutive amino acid, and

(B) identifying the liberated amino acid by immunoassay utilizing an antibody against a derivative of the constitutive amino acid or the constitutive modified amino acid liberated by the aforementioned chemical cutting.

According to the present invention, various types of PTH amino acid including modified PTH amino acid are identified by immunoassay utilizing monoclonal antibodies against the various types of PTH amino acid liberated by Edman degradation. As to the immunoassay, various methods of immunoassay can be utilized in addition to a “competitive method” described with reference to an embodiment of the present invention.

In relation to a method of creating monoclonal antibodies, a molecular biological method such as “phage display” can be utilized in addition to methods generally used of obtaining monoclonal antibodies by immunizing various types of PTH amino acid to mice or the like.

The present invention uses the immunoassay for detection of liberated constitutive amino acid, whereby detection in orders of attomol and zmol is attainable as to detection sensitivity.

Also as to constitutive modified amino acids with saccharides or the like, the present invention can be readily attained when preparing corresponding monoclonal antibodies.

The foregoing and other objects, features, aspects and advantages of the present invention will become more apparent from the following detailed description of the present invention when taken in conjunction with the accompanying drawing.

### BRIEF DESCRIPTION OF THE DRAWINGS

FIGS. 1 A to 1D illustrate immunoassay used in the present invention.

### DESCRIPTION OF THE PREFERRED EMBODIMENTS

The procedure of a method according to the present invention is as follows:

(Step 1): Edman Degradation

An Edman degradation apparatus put into practice at present is utilized as such for performing Edman degradation and obtaining PTH amino acid derivatives.

(Step 2):

Immunoassay is carried out on the PTH amino acid derivatives obtained at the step 1. For example, a competitive method is employed here as the immunoassay. This immunoassay shall now be described.

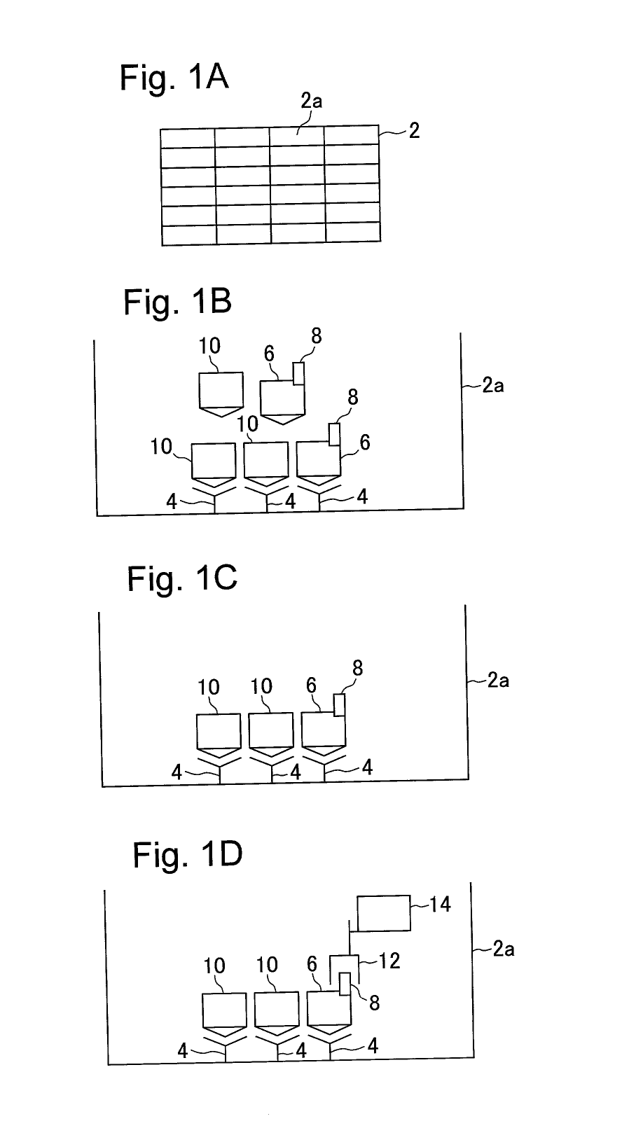

FIG. 1A shows a microplate 2 employed for the immunoassay. Monoclonal antibodies against various types of PTH amino acids are fixed in the respective wells 2a one by one. A certain quantity of analogs (analogous compounds) 6 provided with labels 8 to the PTH amino acid derivatives 10, which bond with the respective monoclonal antibody 4, are previously added to the respective wells 2a to be bonded with the monoclonal antibodies 4 (see the right end of FIG. 1B).

A solution containing the PTH amino acid derivative 10 obtained at the step 1 is dripped on to the microplate 2. Thus, bonding with the monoclonal antibody 4 is competed between the PTH amino acid derivative 10 and the analog 6 in the well 2a to which the monoclonal antibody 4 reacting with the PTH amino acid derivative 10 is fixed, as shown in FIG. 1B.

Next, non-bonded PTH amino acid derivative 10 and the analog 6 liberated by competitive reaction are washed out, thereby leaving the PTH amino acid derivative 10 bonded with the monoclonal antibody 4 and the analog 6 bonded with the monoclonal antibody 4, as shown in FIG. 1C.

Then, an antibody 12 against the analog 6 is added so that the antibody 12 is bonded to the label 8 of the analog 6 as shown in FIG. 1D. For example, enzyme is bonded to the antibody 12 as a label 14. Thereafter a non-bonded antibody 12 is washed out.

Thereafter the quantity of the enzyme-labeled antibody 12 bonded with the label 8 of the analog 6 is measured thereby measuring the quantity of the analog 6.

According to this immunoassay, the quantity of the monoclonal antibodies 4 in the respective wells 2a are fixed, and the PTH amino acid derivative 10 and the analog 6 are competitively bonded to the antibody 4. When the quantity of the analog 6 is also fixed, it follows that the quantity of bonded analog 6 is reduced as the quantity of the PTH amino acid derivative 10 is increased and the quantity of final enzyme-labeled antibody 12 is also reduced. The types and quantities of the PTH amino acid derivatives 10 can be determined according to this principle.

While the above description has been made with reference to the competition method employed as the immunoassay, other immunoassay can alternatively be utilized.

When utilizing fluorescence depolarization or the like as the method of detecting PTH amino acids bonded with antibodies, a washing step or the like inevitable in the above description can be omitted for simplifying the steps.

The flow of the steps of the overall method according to the present invention is as follows:

[Edman degradation from an N end]→[identification of first N end amino acid by immunoassay]→[next Edman degradation]→[identification of second N-end amino acid by immunoassay]→repeat

While the description has been made with reference to a microplate, conversion to a chip can also be readily performed when fixing antibodies to fine regions.

Although the present invention has been described and illustrated in detail, it is clearly understood that the same is by way of illustration and example only and is not to be taken by way of limitation as the spirit and scope of the present invention are limited only by the terms of the appended claims.

What is claimed is:

1. An amino acid sequence determination for protein or peptide including the following steps (A) and (B):
(A) chemically cutting constitutive amino acid one by one from an N end of protein or peptide for liberating the constitutive amino acid; and
(B) identifying the liberated constitutive amino acid by immunoassay utilizing an antibody against a derivative of the constitutive amino acid liberated by the chemical cutting.

2. The amino acid sequence determination according to claim 1, wherein
the constitutive amino acid includes constitutive modified amino acid.

3. The amino acid sequence determination according to claim 1, wherein
the steps (A) and (B) are successively repeated one by one every the constitutive amino acid.

4. The amino acid sequence determination according to claim 1, wherein
the immunoassay is a competitive method.

5. The amino acid sequence determination according to claim 4, wherein
the immunoassay employs such a microplate that monoclonal antibodies for various types of PTH amino acids are fixed to respective wells of the microplate one by one and analogs with labels to PTH amino acid derivatives bonding with the respective monoclonal antibodies are previously added to be bonded with the monoclonal antibodies and includes steps of
(1) dripping a solution containing PTH amino acid derivatives obtained by Edman degradation on to the microplate,
(2) washing out non-bonded PTH amino acid derivatives and analogs liberated by competitive reaction,
(3) adding labeled antibodies against analogs, and
(4) measuring the quantity of the labeled antibodies bonded with the analogs.

6. The amino acid sequence determination according to claim 5, wherein
the quantity of the monoclonal antibodies in the respective wells as well as the quantity of the analogs are fixed.
